(19) United States
(12) Patent Application Publication
(10) Pub. No.: US 2026/0198993 A1
(43) Pub. Date: Jul. 16, 2026

(54) ENDOSCOPIC TREATMENT INSTRUMENT

(71) Applicant: FUJIFILM Corporation, Tokyo (JP)

(72) Inventors: Keisuke USHIDA, Ashigarakami-gun (JP); Ryo ISHIKAWA, Ashigarakami-gun (JP)

(73) Assignee: FUJIFILM Corporation, Tokyo (JP)

(21) Appl. No.: 19/440,648

(22) Filed: Jan. 6, 2026

(30) Foreign Application Priority Data
Jan. 10, 2025 (JP) 2025-004260

(51) Int. Cl. A61B 18/14; A61B 18/00

(52) U.S. Cl. CPC A61B 18/1445; A61B 2018/00172; A61B 2018/00184; A61B 2018/0091; A61B 2018/00982

(57) ABSTRACT

An endoscopic treatment instrument includes: a treatment member including, on a distal end side, a treatment part that is openable and closable and a support part that supports a proximal end side of the treatment part, and extending in a first direction; a housing member that has a cylindrical shape, that extends in the first direction, and into which the treatment member is inserted; a flow path that is formed between an inner peripheral surface of the housing member and the treatment member and through which a liquid supplied from a proximal end side of the housing member to an inner portion of the housing member is capable of being discharged from a distal end side opening of the housing member; and a narrowed part of the flow path, the narrowed part being provided on a distal end side with respect to a proximal end edge of the support part.

### CROSS-REFERENCE TO RELATED APPLICATIONS

The present application claims priority under 35 U.S.C. §119 to Japanese Patent Application No. 2025-004260 filed on January 10, 2025. The above application is hereby expressly incorporated by reference, in its entirety, into the present application.

### BACKGROUND OF THE INVENTION

### Field of the Invention

The present invention relates to an endoscopic treatment instrument.

### Description of the Related Art

JP2024-4890A discloses an endoscopic treatment instrument comprising: a treatment instrument body extending in a first direction and having a distal end portion that is openable and closable; an operation member provided on a proximal end side of the treatment instrument body and used to perform an opening and closing operation of the distal end portion; and a cylindrical housing member that houses the treatment instrument body therein and that has a through-hole through which a fluid is supplied to the inside, in which the housing member and the treatment instrument body are configured to be relatively movable in the first direction such that the housing member and the treatment instrument body can be in a first state in which the treatment instrument body protrudes from a first opening on a distal end side of the housing member, and a second state in which at least a part of a protrusion range of the treatment instrument body in the first state is retracted to a proximal end side of the first opening.

### SUMMARY OF THE INVENTION

An aspect of the technology of the present disclosure relates to an endoscopic treatment instrument comprising: a treatment member including, on a distal end side, a treatment part that is openable and closable and a support part that supports a proximal end side of the treatment part, and extending in a first direction; a housing member that has a cylindrical shape, that extends in the first direction, and into which the treatment member is inserted; a flow path that is formed between an inner peripheral surface of the housing member and the treatment member and through which a liquid supplied from a proximal end side of the housing member to an inner portion of the housing member is capable of being discharged from a distal end side opening of the housing member; and a narrowed part of the flow path, in which the narrowed part is provided on a distal end side with respect to a proximal end edge of the support part.

### BRIEF DESCRIPTION OF THE DRAWINGS

FIG. 1 is a schematic view illustrating a schematic configuration of a system including an endoscopic treatment instrument 1 according to an embodiment of the present invention.

FIG. 2 is a perspective view illustrating a configuration of a treatment member 20.

FIG. 3 is an exploded perspective view of a part of the treatment member 20.

FIG. 4 is an enlarged perspective view of a shaft support member 221.

FIG. 5 is a perspective view of the shaft support member 221 as viewed in a direction different from that in FIG. 3.

FIG. 6 is a schematic cross-sectional view illustrating a detailed configuration example of an operating part 50.

FIG. 7 is a schematic view illustrating a state in which the operating part 50 illustrated in FIG. 6 is operated.

FIG. 8 is a schematic view of a housing member 30 and the treatment member 20 as viewed from above.

FIG. 9 is a schematic view of the housing member 30 and the treatment member 20 as viewed from below.

FIG. 10 is a schematic view of the housing member 30 and the treatment member 20 as viewed from a left side.

FIG. 11 is a schematic view of the housing member 30 and the treatment member 20 as viewed from a right side.

FIG. 12 is a schematic cross-sectional view taken along line A-A illustrated in FIG. 10.

FIG. 13 is a schematic cross-sectional view taken along line B-B illustrated in FIG. 10.

FIG. 14 is a schematic cross-sectional view taken along line C-C illustrated in FIG. 10.

FIG. 15 is a schematic cross-sectional view of a covering member 23E and the operating part 50 in the endoscopic treatment instrument 1.

FIG. 16 is a schematic view of the endoscopic treatment instrument 1 as viewed from a front side.

FIG. 17 is a schematic view of a first modification example of the endoscopic treatment instrument 1 as viewed from a front side.

FIG. 18 is a schematic cross-sectional view of the endoscopic treatment instrument 1 illustrated in FIG. 17, which is taken at a position of an annular part 221C.

FIG. 19 is a schematic cross-sectional view of the endoscopic treatment instrument 1 illustrated in FIG. 17, which is taken at a position of the shaft support member 221.

FIG. 20 is a schematic view of a second modification example of the endoscopic treatment instrument 1 as viewed from a front side.

FIG. 21 is a schematic view of a third modification example of the endoscopic treatment instrument 1 as viewed from a front side.

### DESCRIPTION OF THE PREFERRED EMBODIMENTS

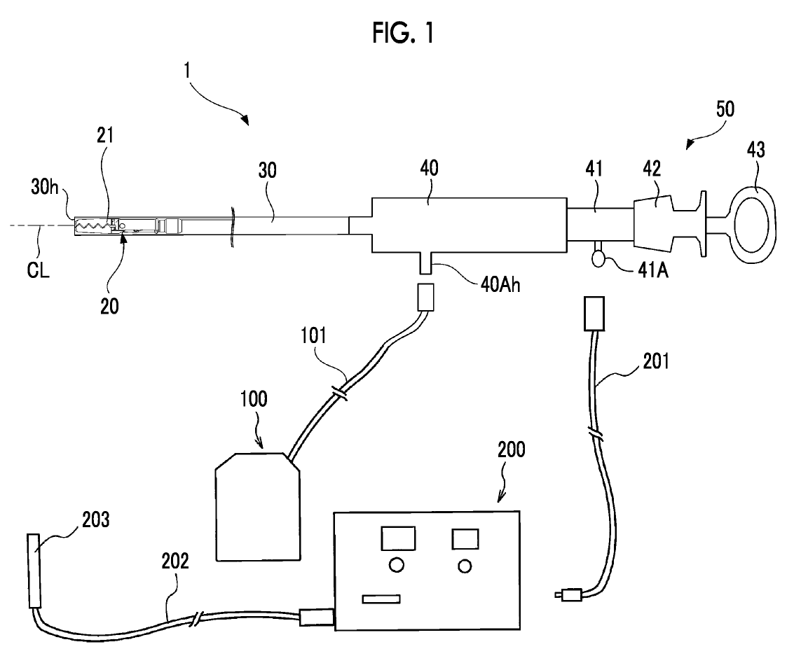

FIG. 1 is a schematic view illustrating a schematic configuration of a system including an endoscopic treatment instrument 1 according to an embodiment of the present invention. The system illustrated in FIG. 1 comprises the endoscopic treatment instrument 1, a liquid supply device 100 that supplies a liquid such as water or a local injection fluid, a supply pipe 101 that connects the endoscopic treatment instrument 1 to the liquid supply device 100, a high-frequency power supply 200, a cable 201 that connects the endoscopic treatment instrument 1 to the high-frequency power supply 200, a counter electrode 203 attached to a treatment target (for example, a human body), and a cable 202 that connects the high-frequency power supply 200 to the counter electrode 203.

The endoscopic treatment instrument 1 is used by being inserted into a treatment instrument insertion channel of an endoscope (not illustrated). Hereinafter, in the endoscopic treatment instrument 1 in a state of being inserted into the treatment instrument insertion channel, an outlet side of the treatment instrument insertion channel is referred to as a distal end side, and an inlet side of the treatment instrument insertion channel is referred to as a proximal end side. A direction along an axis of the treatment instrument insertion channel of the endoscope is referred to as an axial direction. The axial direction constitutes a first direction. This axial direction is the same as a direction along an axis CL of the endoscopic treatment instrument 1.

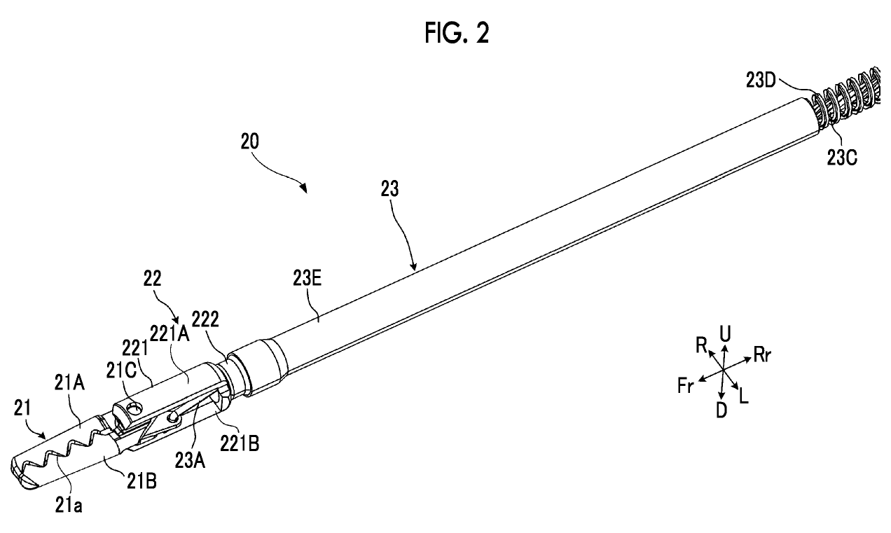

The endoscopic treatment instrument 1 comprises a treatment member 20 having, on the distal end side, a treatment part 21 that is openable and closable and a support part 22 (see FIG. 2) that supports the proximal end side of the treatment part 21, and extending in the axial direction, a housing member 30 that has a cylindrical shape, that extends in the axial direction, and into which the treatment member 20 is inserted, and an operating part 50 that is provided on the proximal end side of the housing member 30 and that performs an opening and closing operation of the treatment part 21.

The housing member 30 is flexible and is inserted into the treatment instrument insertion channel of the endoscope.

The operating part 50 comprises a distal end member 40 that is fixed to a proximal end of the housing member 30, a cable connection part 41 that is provided on the proximal end side of the distal end member 40 and that has a connection terminal 41A for electrically connecting to the cable 201, a slider 42 that is provided on the proximal end side of the cable connection part 41, and a finger rest part 43 that is provided on the proximal end side of the slider 42. A liquid supply part 40Ah that is connected to the supply pipe 101 is provided in the distal end member 40.

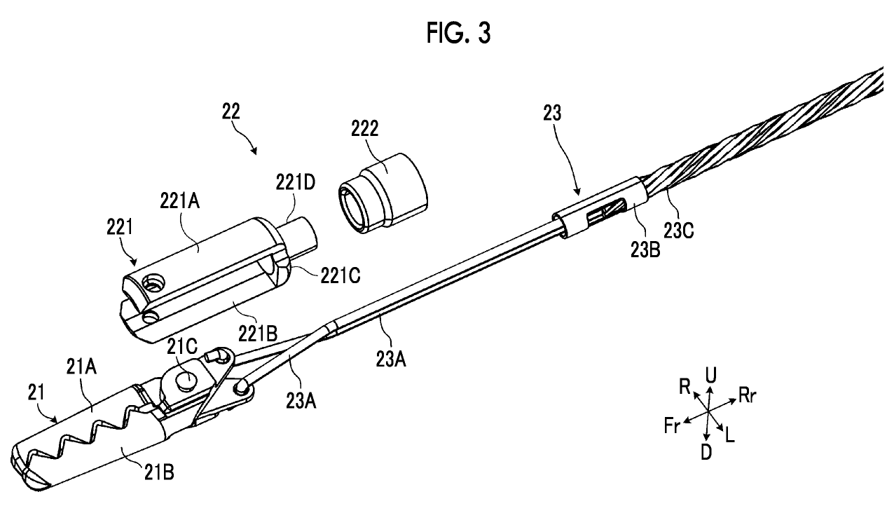

FIG. 2 is a perspective view illustrating a configuration of the treatment member 20. FIG. 3 is an exploded perspective view of a part of the treatment member 20.

As illustrated in FIG. 2, the treatment member 20 comprises the treatment part 21 that is provided on the distal end side, the support part 22 that supports the proximal end side of the treatment part 21, and a connection part 23 that connects the treatment part 21 to the operating part 50.

The treatment part 21 comprises a pair of claw parts 21A and 21B that have serrations 21a on an inner side and that are openable and closable around an opening and closing shaft 21C that extends in a direction perpendicular to the axial direction.

FIG. 2 illustrates a front direction Fr that is a direction from the proximal end side to the distal end side of the endoscopic treatment instrument 1, and a rear direction Rr that is a direction opposite to the front direction Fr. FIG. 2 illustrates a right direction R that is one side of the opening and closing direction of the claw parts 21A and 21B, and a left direction L that is the other side of the opening and closing direction. FIG. 2 illustrates an up direction U that is one side of a direction perpendicular to the opening and closing direction of the claw parts 21A and 21B, and a down direction D that is the other side of the perpendicular direction.

The front direction Fr and the rear direction Rr are collectively referred to as a front-rear direction. The front-rear direction is synonymous with the above-described axial direction. The right direction R and the left direction L are collectively referred to as a left-right direction. The up direction U and the down direction D are collectively referred to as an up-down direction. The front-rear direction, the left-right direction, and the up-down direction intersect each other.

As illustrated in FIG. 3, the support part 22 comprises a shaft support member 221 that supports the opening and closing shaft 21C of the treatment part 21, and a coupling member 222 that couples the shaft support member 221 to the connection part 23. The shaft support member 221 is substantially U-shaped as viewed in the left-right direction. The coupling member 222 has a cylindrical shape in which the axis extends in the front-rear direction.

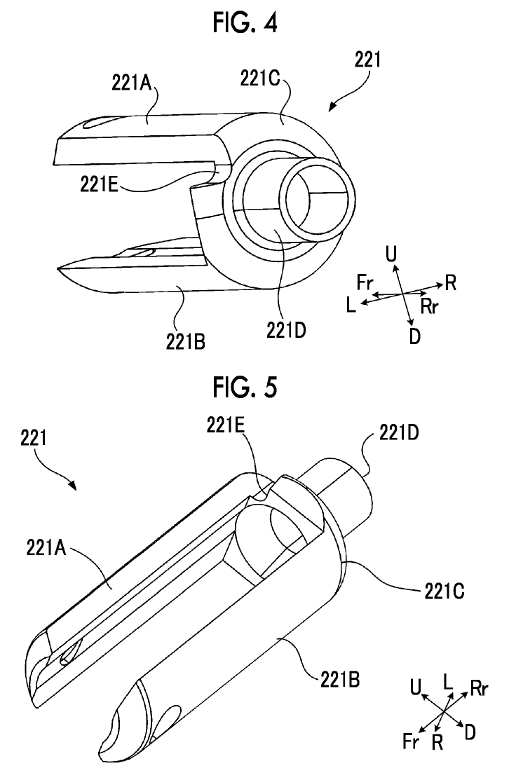

FIG. 4 is an enlarged perspective view of the shaft support member 221. FIG. 5 is a perspective view of the shaft support member 221 as viewed from a direction different from that in FIG. 4.

As illustrated in FIGS. 4 and 5, the shaft support member 221 comprises an upper shaft support part 221A and a lower shaft support part 221B that are arranged in the up-down direction and that support the opening and closing shaft 21C on the distal end side, an annular part 221C that is annular as viewed in the front-rear direction and that is provided on proximal end sides of the upper shaft support part 221A and the lower shaft support part 221B, and a cylindrical extension part 221D that extends from a periphery of an opening on the proximal end side of the annular part 221C to the proximal end side.

The upper shaft support part 221A and the lower shaft support part 221B each have a thickness in the up-down direction and are formed in a shape of a curved plate that is curved in a direction away from the axis CL. The extension part 221D is configured to have a smaller diameter than the annular part 221C.

As illustrated in FIG. 2, the proximal end sides of the claw parts 21A and 21B are disposed between the upper shaft support part 221A and the lower shaft support part 221B and are supported therebetween.

As illustrated in FIG. 3, the support part 22 further comprises the coupling member 222 that couples the shaft support member 221 to the connection part 23. The coupling member 222 is formed in a cylindrical shape that extends in the front-rear direction. The extension part 221D of the shaft support member 221 is inserted from the distal end side into the coupling member 222, and the coupling member 222 and the extension part 221D are fixed to each other inside the coupling member 222.

The connection part 23 comprises a pair of link rods 23A of which distal end sides are supported by the claw parts 21A and 21B, an operation wire 23C that extends from the operating part 50 to positions of proximal ends of the link rods 23A, and a cylindrical member 23B that supports a distal end of the operation wire 23C inside. The pair of link rods 23A protrude from between the upper shaft support part 221A and the lower shaft support part 221B to the proximal end side of the support part 22 through the inside of the annular part 221C, the extension part 221D, and the coupling member 222. The link rods 23A are inserted into the cylindrical member 23B and are fixed to a distal end surface of the operation wire 23C inside the cylindrical member 23B.

As illustrated in FIG. 2, the connection part 23 comprises a coil sleeve 23D that consists of a tightly wound coil provided around an outer periphery of the cylindrical member 23B and the operation wire 23C on the proximal side of the link rods 23A with respect to the support part 22, and a covering member 23E that covers the distal end side of the coil sleeve 23D. A distal end of the coil sleeve 23D is fixed inside the coupling member 222. A proximal end of the coil sleeve 23D is fixed inside the distal end member 40 illustrated in FIG. 1. The operation wire 23C moves in the front-rear direction, and a link mechanism using the pair of link rods 23A is operated to open and close the claw parts 21A and 21B.

As illustrated in FIG. 5, a gap is formed between both ends of the upper shaft support part 221A in the left-right direction and both ends of the lower shaft support part 221B in the left-right direction in order to prevent interference with the proximal end sides of the claw parts 21A and 21B or the link rods 23A when the claw parts 21A and 21B are opened or closed.

As illustrated in FIGS. 4 and 5, the annular part 221C is provided with a groove 221E extending in the front-rear direction in a part of a circumferential direction.

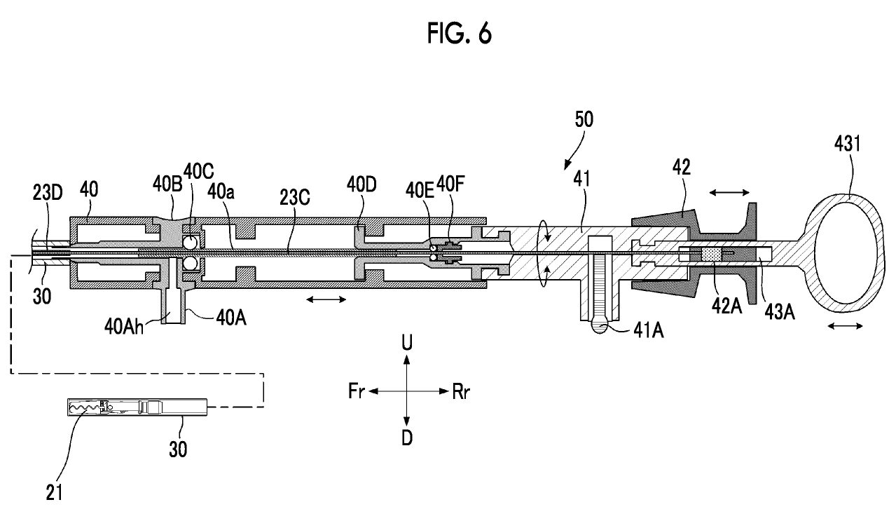

FIG. 6 is a schematic cross-sectional view illustrating a detailed configuration example of the operating part 50. The distal end member 40 has a cylindrical shape and is fixed to the housing member 30 at the distal end, and thus the inside of the housing member 30 communicates with the inside of the distal end member 40.

A connector member 40A, which is fixed to the distal end member 40, is provided inside the distal end member 40. The connector member 40A has a generally T-shaped cross section and includes a first linear portion extending in the up-down direction and a second linear portion extending from approximately the center of the first linear portion in the up-down direction toward the distal end side. A through-hole that penetrates in the front-rear direction is provided in the second linear portion, and the through-hole communicates with the inside of the housing member 30 on the distal end side and communicates with the inside of the distal end member 40 on the proximal end side. The liquid supply part 40Ah formed by a hole allowing the through-hole to communicate with the outside is provided inside a lower portion of the through-hole in the first linear portion of the connector member 40A. In a case in which the liquid is delivered from the liquid supply device 100 in a state in which the supply pipe 101 is connected to the liquid supply part 40Ah, the liquid is supplied to the housing member 30 via the through-hole of the connector member 40A. The distal end member 40 and the connector member 40A constitute a liquid supply part that supplies a liquid from the proximal end side to the housing member 30.

An O-ring 40C as a sealing member provided on the proximal end side of the connector member 40A and a cylindrical connector member 40D with a flange provided on the proximal end side of the O-ring 40C are provided inside the distal end member 40. The O-ring 40C is sandwiched between an inner wall of the distal end member 40 and an outer surface of the connector member 40A on the proximal end side. A cylindrical member 40a extending to the distal end side is fixed inside the distal end side of the connector member 40D. The cylindrical member 40a extends to the inside of the connector member 40A.

In the connection part 23 of the treatment member 20, the proximal end sides of the operation wire 23C and the coil sleeve 23D are provided inside the distal end member 40. The coil sleeve 23D extends from a position of the coupling member 222 to a position of the distal end side of the connector member 40A. The operation wire 23C extends further to the proximal end side with respect to the proximal end of the coil sleeve 23D, and extends to the proximal end side with respect to the connector member 40D through the inside of the cylindrical member 40a. The connector member 40D is configured to be movable in the front-rear direction relative to the distal end member 40 and the connector member 40A.

An O-ring 40E as a sealing member is provided on an inner periphery of the connector member 40D between the connector member 40D and the operation wire 23C. The O-ring 40C and the O-ring 40E are provided on the proximal end side with respect to an inner space of the connector member 40A into which the liquid flows from the liquid supply part 40Ah. The O-ring 40C and the O-ring 40E firmly prevent the liquid supplied to the inside of the connector member 40A from moving to a space on the proximal end side with respect to the connector member 40A in the distal end member 40. A fixing member 40F for fixing the O-ring 40E is provided on the proximal end side of the O-ring 40E inside the connector member 40D.

The cable connection part 41 is composed of a cylindrical member into which the operation wire 23C extending from the connector member 40D to the proximal end side is inserted, and the distal end side of the cable connection part 41 is supported by a proximal end portion of the connector member 40D. The cable connection part 41 and the connector member 40D are coupled to be non-movable relative to each other in the front-rear direction, and are coupled to be rotatable relative to each other around the axis CL. The connection terminal 41A is electrically connected to the operation wire 23C inserted into the cable connection part 41.

The finger rest part 43 is supported inside the cable connection part 41 on the proximal end side. The cable connection part 41 and the finger rest part 43 are coupled to be non-movable relative to each other in the front-rear direction, and are coupled to be rotatable relative to each other around the axis CL.

The slider 42 is provided on outer peripheries of the cable connection part 41 and the finger rest part 43 across the cable connection part 41 and the finger rest part 43, and is configured to be movable in the front-rear direction with respect to the cable connection part 41 and the finger rest part 43. The slider 42 and the cable connection part 41 are coupled to be rotatable relative to each other around the axis CL. The slider 42 and the finger rest part 43 are coupled to be non-rotatable relative to each other around the axis CL. The operation wire 23C inserted from the distal end side into the cable connection part 41 is inserted into the finger rest part 43 and is fixed to a wire fixing part 42A of the slider 42 in a cavity 43A inside the finger rest part 43.

The endoscopic treatment instrument 1 is configured to be capable of being in a protruding state in which the treatment part 21 protrudes from a distal end side opening 30h of the housing member 30 and a retracted state in which the entire protrusion range of the treatment part 21 from the distal end side opening 30h in the protruding state is retracted to the proximal end side with respect to the distal end side opening 30h. In the protruding state, the treatment part 21 protrudes from the distal end side opening 30h to the extent that the treatment part 21 can be opened to its maximum. FIG. 6 illustrates the retracted state.

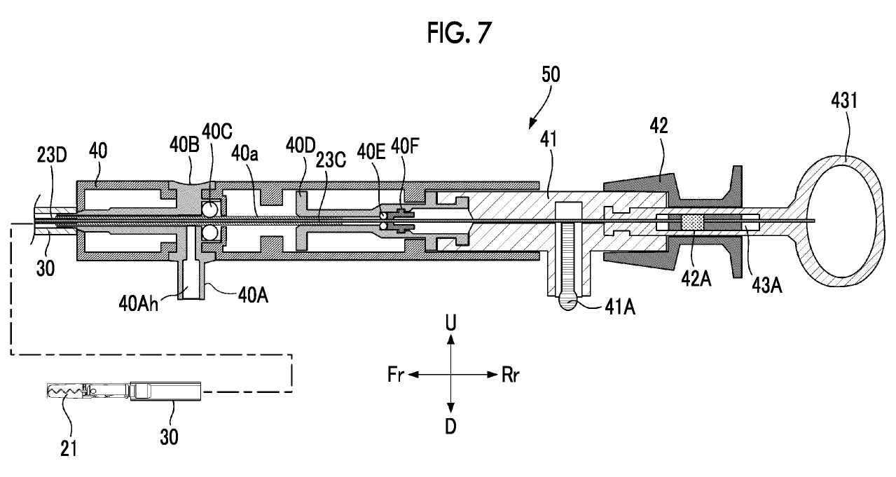

In a case of using the endoscopic treatment instrument 1, in the retracted state illustrated in FIG. 6, for example, an operation of moving the distal end member 40 to the proximal end side while holding a position of the finger rest part 43 in the front-rear direction is performed. As a result of this operation, as illustrated in FIG. 7, the distal end member 40, the connector member 40A, and the O-ring 40C move to the proximal end side with respect to the cylindrical member 40a, the connector member 40D, and the cable connection part 41. As a result, although the position of the treatment member 20 in the front-rear direction does not change, the distal end of the housing member 30 moves to the proximal end side, and thus the protruding state is obtained.

In the protruding state, in a case in which the slider 42 is moved in the front-rear direction with respect to the cable connection part 41 and the finger rest part 43, the operation wire 23C moves in the front-rear direction. By moving the operation wire 23C to open and close the treatment part 21 and to grip and energize a submucosal layer or the like, a lesion can be excised.

A doctor opens and closes the treatment part 21 by means of the operating part 50 to excise the lesion with the treatment part 21, and then moves, for example, the housing member 30 and the distal end member 40 to the distal end side to house the entire treatment part 21 in the housing member 30. Thereafter, the doctor pulls out the endoscopic treatment instrument 1 from the endoscope. Through the above-described procedures, a treatment such as endoscopic submucosal dissection (ESD) is performed.

In the protruding state, in a case in which a rotation operation of rotating the finger rest part 43 around the axis CL is performed, the slider 42 rotates together with the finger rest part 43, and the operation wire 23C fixed to the wire fixing part 42A of the slider 42 rotates. In a case in which the operation wire 23C rotates, the link rods 23A fixed to the operation wire 23C also rotate. Therefore, the treatment part 21 can be rotated in the protruding state to flexibly change an orientation of the treatment part 21.

The cable connection part 41 can rotate with respect to the connector member 40D, the slider 42, and the finger rest part 43. Therefore, for example, in a case in which a rotation operation of rotating the finger rest part 43 in a state of gripping the distal end member 40 is performed, the cable connection part 41 can be prevented from following the rotation. Since the cable 201 is connected to the cable connection part 41, the cable connection part 41 does not rotate, and thus the cable 201 does not rotate either, and treatment using the endoscopic treatment instrument 1 can be performed efficiently.

In addition, even in a case in which it is desired to change the position of the cable 201, the cable connection part 41 can rotate with respect to the connector member 40D, so that the position of the cable 201 can be changed while the position of the distal end member 40 is maintained.

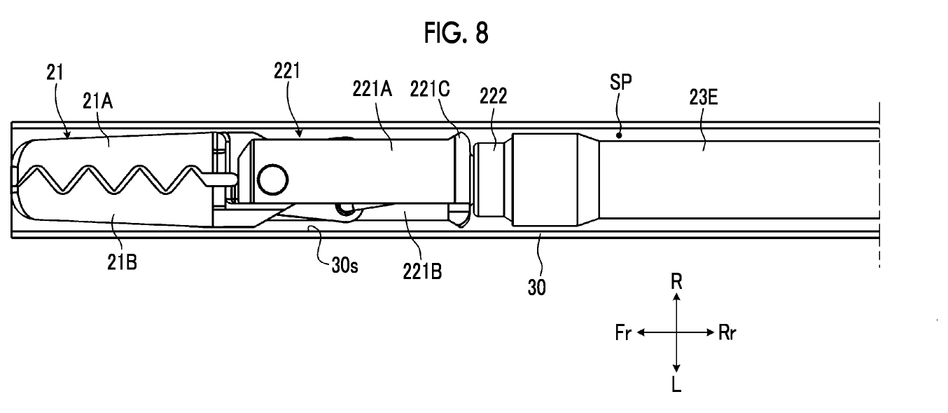

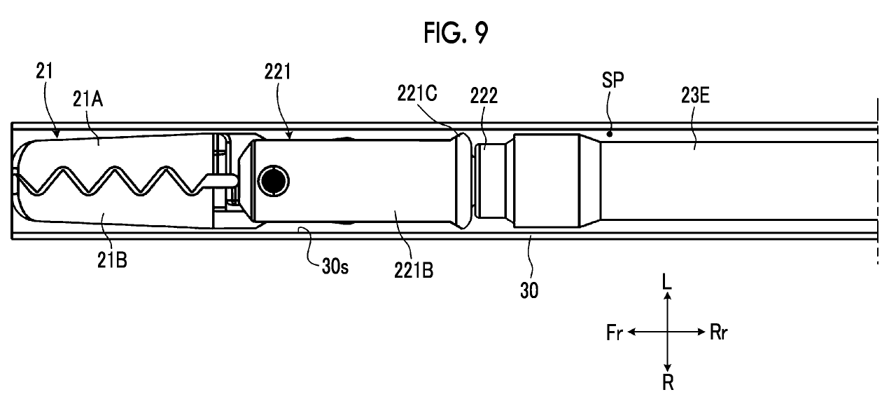

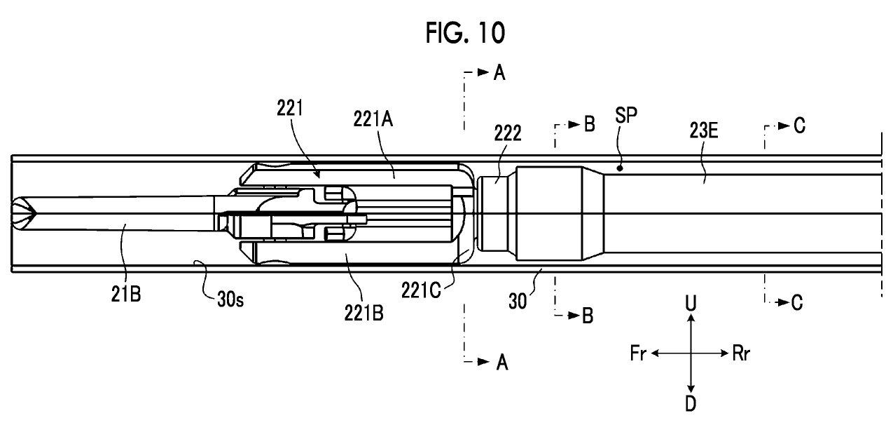

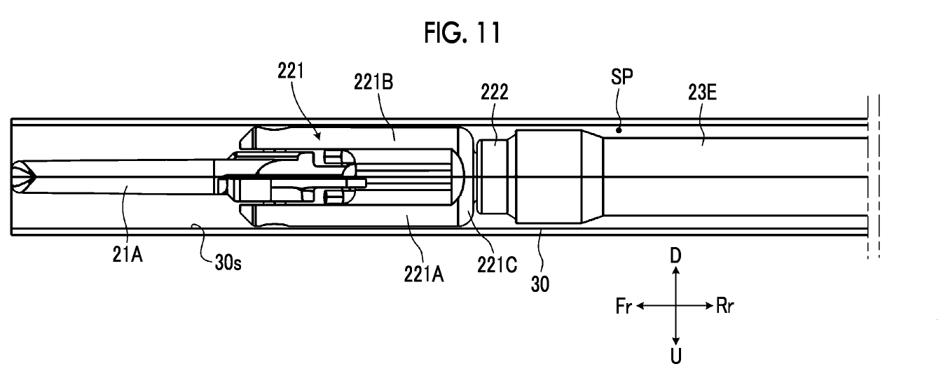

FIG. 8 is a schematic view of the housing member 30 and the treatment member 20 as viewed from above. FIG. 9 is a schematic view of the housing member 30 and the treatment member 20 as viewed from below. FIG. 10 is a schematic view of the housing member 30 and the treatment member 20 as viewed from a left side. FIG. 11 is a schematic view of the housing member 30 and the treatment member 20 as viewed from a right side. In FIGS. 8 to 11, the housing member 30 is illustrated in cross section.

As illustrated in FIGS. 8 to 11, a flow path SP through which the liquid supplied from the proximal end side of the housing member 30 to the inside of the housing member 30 by the distal end member 40 can be discharged from the distal end side opening 30h of the housing member 30 is formed between an inner peripheral surface 30s of the housing member 30 and the treatment member 20.

A cross-sectional area of the flow path SP in a cross section perpendicular to the front-rear direction varies depending on the position of the housing member 30 in the front-rear direction. In a range from the proximal end to the distal end side of the housing member 30, the cross-sectional area is large since a diameter of a structure housed in the housing member 30 is small. As a result, the liquid can smoothly flow from the proximal end side to the distal end side.

A large structure such as the shaft support member 221 or the coupling member 222 is provided on the distal end side of the housing member 30. Therefore, the cross-sectional area of the flow path SP tends to be small on the distal end side of the housing member 30.

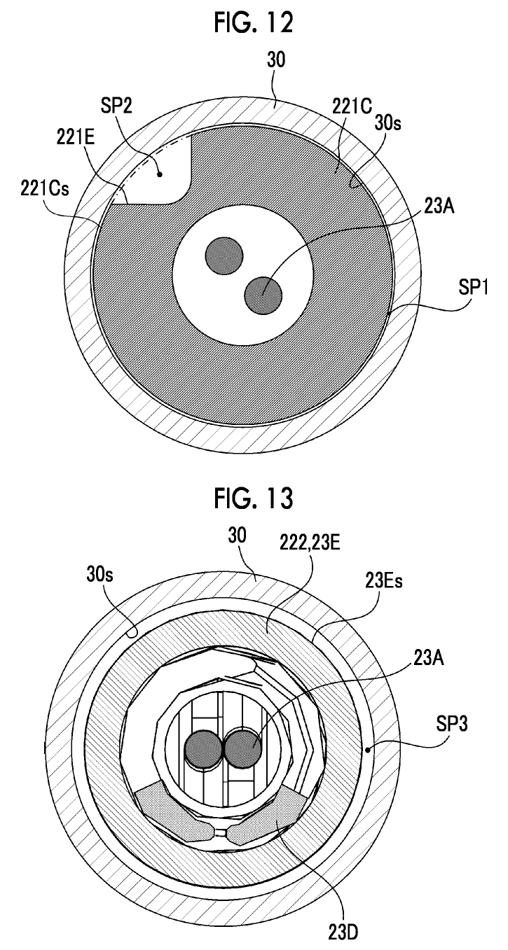

In the present embodiment, the cross-sectional area of the flow path SP is configured to be minimized at a position of the annular part 221C in the front-rear direction. FIG. 12 is a schematic cross-sectional view taken along line A-A illustrated in FIG. 10. As illustrated in FIG. 12, at the position of the annular part 221C, the flow path SP is a combination of a gap SP1 between an outer peripheral surface 221Cs of the annular part 221C excluding the groove 221E and the inner peripheral surface 30s of the housing member 30 and a gap SP2 between the groove 221E and the inner peripheral surface 30s.

The gap SP1 is the minimum distance that allows the treatment member 20 and the housing member 30 to move relative to each other in the front-rear direction, and is small enough that a fluid cannot substantially pass through. Therefore, at the position of the annular part 221C, the gap SP2 can be regarded as the flow path SP.

FIG. 13 is a schematic cross-sectional view taken along line B-B illustrated in FIG. 10. As illustrated in FIG. 13, in a portion in which the coupling member 222 and the covering member 23E overlap each other, the flow path SP consists of a gap SP3 between an outer peripheral surface 23Es of the covering member 23E and the inner peripheral surface 30s of the housing member 30. A cross-sectional area of the gap SP3 is, for example, 4.7 times a cross-sectional area of the gap SP2 illustrated in FIG. 12. In a case of being viewed in the front-rear direction, most of the gap SP3 is covered with the annular part 221C illustrated in FIG. 12.

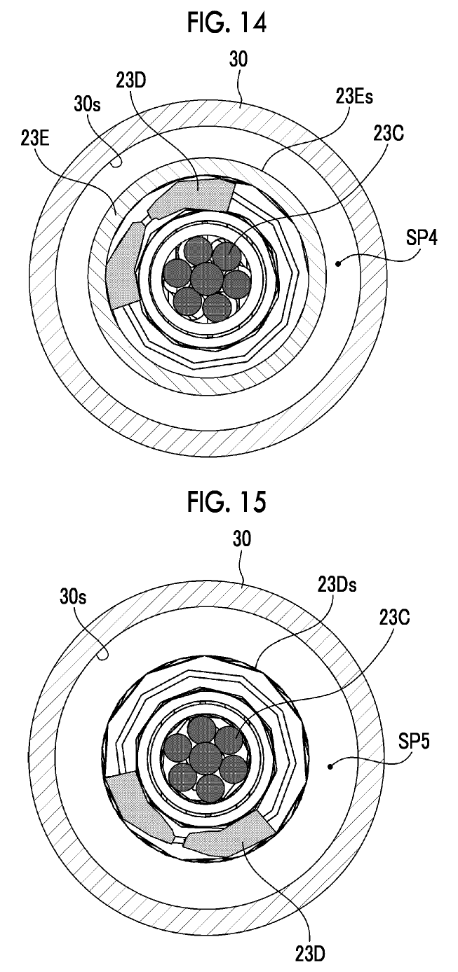

FIG. 14 is a schematic cross-sectional view taken along line C-C illustrated in FIG. 10. As illustrated in FIG. 14, in a portion of the covering member 23E on the proximal end side with respect to the coupling member 222, the flow path SP consists of a gap SP4 between the outer peripheral surface 23Es of the covering member 23E and the inner peripheral surface 30s of the housing member 30. A cross-sectional area of the gap SP4 is, for example, 10.4 times the cross-sectional area of the gap SP2 illustrated in FIG. 12. In a case of being viewed in the front-rear direction, most of the gap SP4 is covered with the annular part 221C illustrated in FIG. 12.

FIG. 15 is a schematic cross-sectional view of the proximal end side with respect to the covering member 23E in the endoscopic treatment instrument 1. As illustrated in FIG. 15, in a portion between the covering member 23E and the operating part 50, the flow path SP consists of a gap SP5 between an outer peripheral surface 23Ds of the coil sleeve 23D and the inner peripheral surface 30s of the housing member 30. A cross-sectional area of the gap SP5 is, for example, 13.2 times the cross-sectional area of the gap SP2 illustrated in FIG. 12. In a case of being viewed in the front-rear direction, most of the gap SP5 is covered with the annular part 221C illustrated in FIG. 12.

In addition, on the distal end side with respect to the annular part 221C, since a space is present between the lower shaft support part 221B and the upper shaft support part 221A or the treatment part 21 has a flat shape, the cross-sectional area of the flow path SP is larger than the gap SP2.

As described above, in the endoscopic treatment instrument 1, the flow path SP is narrow at the position of the annular part 221C, and the outer peripheral surface 221Cs of the annular part 221C and the groove 221E form a narrowed part of the flow path SP.

The liquid supplied from the proximal end side to the housing member 30 passes through the gap SP5 illustrated in FIG. 15, the gap SP4 illustrated in FIG. 14, and the gap SP3 illustrated in FIG. 13 in this order, and then reaches the gap SP2 illustrated in FIG. 12. Since the gap SP2 has the minimum cross-sectional area, the liquid is compressed in a narrow space of the gap SP2 and is then discharged to the distal end side. Therefore, the liquid delivery pressure can be increased at the position of the annular part 221C.

In a case in which the treatment part 21 that is openable and closable is provided as in the endoscopic treatment instrument 1, a proportion of the treatment part 21 to an inner cross-sectional area of the housing member 30 is reduced as viewed in the front-rear direction, and thus the flow path SP becomes larger around the treatment part 21. In addition, the claw parts 21A and 21B and the shaft support member 221 that pivotally supports the claw parts 21A and 21B to be openable and closable are required, and the distal end side is long. Therefore, the farther from the distal end the position at which the flow path SP is narrowed is, the longer the distance that liquid discharged from that position travels to exit the distal end side opening 30h, making it difficult to increase the liquid delivery pressure even in a case in which the flow path SP is narrowed. According to the present embodiment, since the flow path SP is narrowed by the annular part 221C, the liquid delivery pressure can be increased.

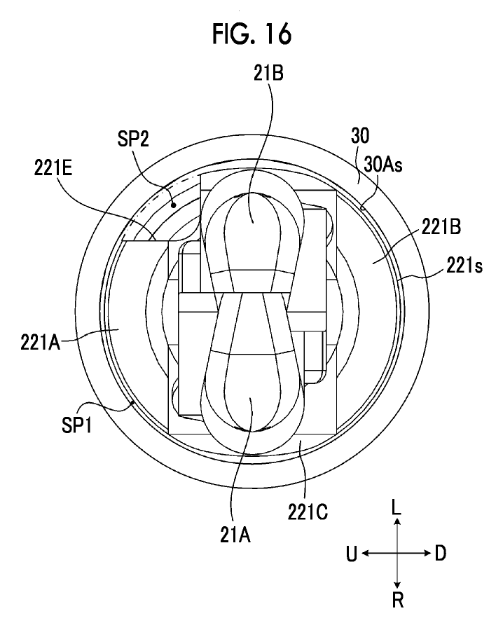

FIG. 16 is a schematic view of the endoscopic treatment instrument 1 as viewed from a front side. As illustrated in FIG. 16, the liquid is delivered from the gap SP2, but other members do not overlap the gap SP2. Therefore, the liquid delivered from the gap SP2 advances to the distal end side along the groove 221E and is delivered forcefully from the distal end side opening 30h. As described above, by narrowing the flow path SP that is annular as in the gap SP3, the gap SP4, and the gap SP5, as a non-annular region, the liquid delivery pressure can be further increased.

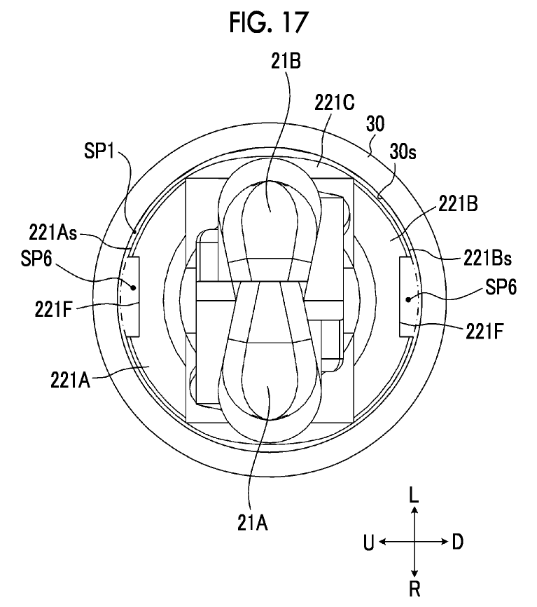

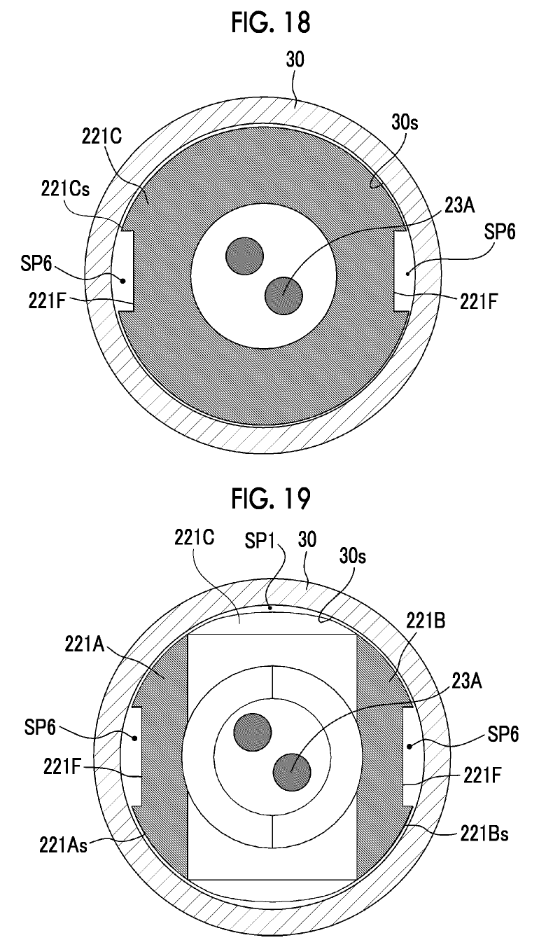

FIG. 17 is a schematic view of a first modification example of the endoscopic treatment instrument 1 as viewed from a front side. FIG. 18 is a schematic cross-sectional view of the endoscopic treatment instrument 1 illustrated in FIG. 17, which is taken at the position of the annular part 221C. FIG. 19 is a schematic cross-sectional view of the endoscopic treatment instrument 1 illustrated in FIG. 17, which is taken at the position of the shaft support member 221.

In the modification example illustrated in FIG. 17, a groove 221F extending in the front-rear direction is provided on an outer peripheral surface 221As of the upper shaft support part 221A, and a groove 221F extending in the front-rear direction is provided on an outer peripheral surface 221Bs of the lower shaft support part 221B. As illustrated in FIG. 18, the groove 221F is also provided in the annular part 221C. In the modification example illustrated in FIG. 17, the gap between the outer peripheral surface (the outer peripheral surface 221Cs of the annular part 221C, the outer peripheral surface 221As of the upper shaft support part 221A, and the outer peripheral surface 221Bs of the lower shaft support part 221B) of the shaft support member 221 and the inner peripheral surface 30s of the housing member 30 is a minimum distance that allows the treatment member 20 and the housing member 30 to be movable relative to each other in the front-rear direction. Therefore, in a case of being viewed in the front-rear direction, most of the gap SP3, the gap SP4, and the gap SP5 are covered with the annular part 221C, the upper shaft support part 221A, and the lower shaft support part 221B.

The groove 221F is formed in the shaft support member 221, so that the liquid can pass through a gap SP6 between the groove 221F and the inner peripheral surface 30s. Therefore, the gap SP6 constitutes the flow path SP, and the cross-sectional area of the gap SP6 is the minimum. In the endoscopic treatment instrument 1 illustrated in FIG. 17, the flow path SP is narrowest at the positions of the annular part 221C, the upper shaft support part 221A, and the lower shaft support part 221B, and the outer peripheral surface 221Cs of the annular part 221C, the outer peripheral surface 221As of the upper shaft support part 221A, the outer peripheral surface 221Bs of the lower shaft support part 221B, and the groove 221F constitute the narrowed part of the flow path SP.

The liquid supplied from the proximal end side to the housing member 30 passes through the gap SP5 illustrated in FIG. 15, the gap SP4 illustrated in FIG. 14, and the gap SP3 illustrated in FIG. 13 in this order, and then reaches the gap SP6 illustrated in FIGS. 18 and 19. Since the gap SP6 has the minimum cross-sectional area, the liquid is compressed in a narrow space of the gap SP6 and is then discharged to the distal end side. Therefore, the liquid delivery pressure can be increased.

As illustrated in FIG. 17, the liquid is delivered from the gap SP6, but other members do not overlap the gap SP6. Therefore, the liquid delivered from the gap SP6 advances to the distal end side along the groove 221F and is delivered forcefully from the distal end side opening 30h. As described above, with the configuration illustrated in FIG. 17 as well, the liquid delivery pressure can be increased.

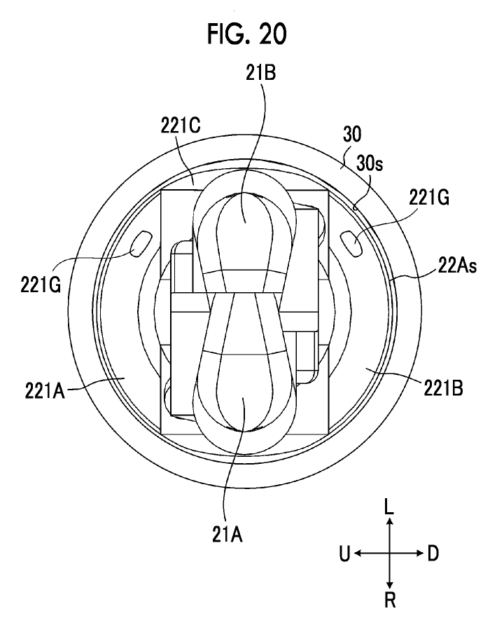

FIG. 20 is a schematic view of a second modification example of the endoscopic treatment instrument 1 as viewed from a front side. In the example illustrated in FIG. 20, a cross section of the annular part 221C is a cross section in which the groove 221E illustrated in FIG. 12 is filled. In addition, in the example illustrated in FIG. 20, the gap between the outer peripheral surface (the outer peripheral surface 221Cs of the annular part 221C, the outer peripheral surface 221As of the upper shaft support part 221A, and the outer peripheral surface 221Bs of the lower shaft support part 221B) of the shaft support member 221 and the inner peripheral surface 30s of the housing member 30 is a minimum distance that allows the treatment member 20 and the housing member 30 to be movable relative to each other in the front-rear direction. In addition, a hole 221G extending from the proximal end surface of the annular part 221C to the distal end surfaces of the upper shaft support part 221A and the lower shaft support part 221B is formed in the shaft support member 221. In this modification example, the narrowed part of the flow path SP is formed by the outer peripheral surface of the shaft support member 221 and the hole 221G. With such a configuration as well, the liquid delivery pressure can be increased.

In addition, with the configurations illustrated in FIGS. 17 and 20, the position at which the cross-sectional area of the flow path SP is minimized can be brought to the position of the distal end of the shaft support member 221. Therefore, the liquid delivery pressure can be further increased. In addition, since only the treatment part 21 is present in a path through which the liquid is delivered from the hole 221G, the liquid is less likely to collide with an obstacle, and the liquid delivery pressure can be increased.

As described so far, the flow path SP is narrowed by providing the groove 221E, the groove 221F, or the hole 221G in the shaft support member 221 while bringing the outer peripheral surface of the shaft support member 221 close to the inner peripheral surface 30s of the housing member 30, but the technology of the present disclosure is not limited to this.

For example, in the coupling member 222, the diameter of a portion exposed from the covering member 23E or a portion covered with the covering member 23E may be increased, the distance between the outer peripheral surface of the diameter-increased portion and the inner peripheral surface 30s may be set to a minimum value at which the treatment member 20 is movable, and the narrowed part of the flow path SP may be formed by providing a groove in the outer peripheral surface of the diameter-increased portion or a through-hole extending in the front-rear direction in the diameter-increased portion.

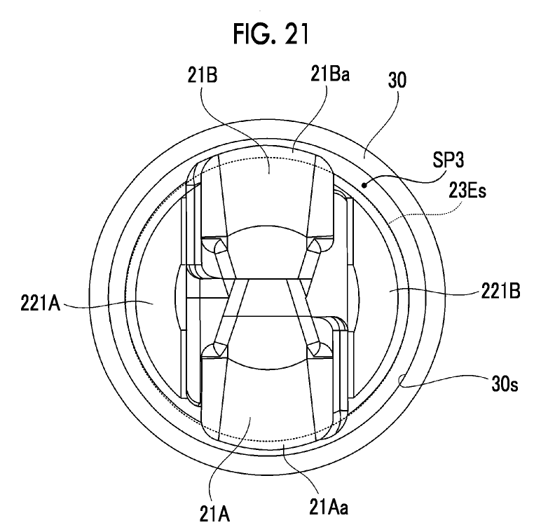

Alternatively, the narrowed part of the flow path SP may be formed by enlarging the treatment part 21. An example of this configuration will be described with reference to FIG. 21.

FIG. 21 is a schematic view of a third modification example of the endoscopic treatment instrument 1 as viewed from a front side. In the example illustrated in FIG. 21, the cross section of the annular part 221C is a cross section in which the groove 221E illustrated in FIG. 12 is filled and an outer diameter is further reduced. In addition, a distance between the outer peripheral surface of the shaft support member 221 and the inner peripheral surface 30s of the housing member 30 is the same as a distance between a portion having a maximum diameter of the covering member 23E and the inner peripheral surface 30s. Therefore, as illustrated in FIG. 21, in a state of being viewed from the front side, a state in which the gap SP3 is visible is obtained.

In the example of FIG. 21, the claw part 21A includes an overlapping part 21Aa that overlaps the gap SP3 as viewed from a front side, and the claw part 21B includes an overlapping part 21Ba that overlaps the gap SP3 as viewed from a front side. In the flow path SP formed by the gap SP3, a portion thereof is closed by the overlapping part 21Aa and the overlapping part 21Ba, and the flow path SP is narrowest (has a minimum cross-sectional area) at the positions of the overlapping part 21Aa and the overlapping part 21Ba. As described above, even in a case in which the narrowed part of the flow path SP is formed by the overlapping part 21Aa and the overlapping part 21Ba by enlarging the treatment part 21, the liquid delivery pressure can be increased.

As described above, at least the following matters are set forth in the present specification.

(1)

An endoscopic treatment instrument comprising: a treatment member including, on a distal end side, a treatment part that is openable and closable and a support part that supports a proximal end side of the treatment part, and extending in a first direction; a housing member that has a cylindrical shape, that extends in the first direction, and into which the treatment member is inserted; a flow path that is formed between an inner peripheral surface of the housing member and the treatment member and through which a liquid supplied from a proximal end side of the housing member to an inner portion of the housing member is capable of being discharged from a distal end side opening of the housing member; and a narrowed part of the flow path, in which the narrowed part is provided on a distal end side with respect to a proximal end edge of the support part.

(2)

The endoscopic treatment instrument according to (1), in which the narrowed part is formed by the support part.

(3)

The endoscopic treatment instrument according to (2), further comprising: an operating part that is provided on a proximal end side and that performs an opening and closing operation of the treatment part, in which the treatment member includes a connection part that connects the treatment part to the operating part, and the support part includes a shaft support member that supports an opening and closing shaft of the treatment part and a coupling member that couples the shaft support member to the connection part.

(4)

The endoscopic treatment instrument according to (3), in which the narrowed part is formed by the shaft support member.

(5)

The endoscopic treatment instrument according to (4), in which the shaft support member has an outer peripheral surface facing the inner peripheral surface of the housing member, the shaft support member is provided with a hole or a groove that extends in the first direction, the shaft support member covers a part of the flow path on a proximal end side with respect to the shaft support member as viewed in the first direction from a distal end side, and the narrowed part is formed by the outer peripheral surface and the hole or the groove of the shaft support member.

(6)

The endoscopic treatment instrument according to (5), in which the shaft support member includes shaft support parts that are arranged in a direction intersecting an opening and closing direction of the treatment part and that support the opening and closing shaft of the treatment part, and an annular part that is provided at proximal ends of the shaft support parts, and the hole or the groove is provided in the annular part.

(7)

The endoscopic treatment instrument according to (5), in which the shaft support member includes shaft support parts that are arranged in a direction intersecting an opening and closing direction of the treatment part and that support the opening and closing shaft of the treatment part, and an annular part that is provided at proximal ends of the shaft support parts, and the hole or the groove is provided in the shaft support part.

(8)

The endoscopic treatment instrument according to (3), in which the narrowed part is formed by the coupling member.

(9)

The endoscopic treatment instrument according to (1), in which the narrowed part is formed by the treatment part.

(10)

The endoscopic treatment instrument according to (9), in which the treatment part includes a claw part, the claw part has an overlapping part that overlaps the flow path on a proximal end side with respect to the claw part as viewed in the first direction from a distal end side, and the narrowed part is formed by the overlapping part.

(11)

The endoscopic treatment instrument according to any one of (1) to (10), in which a cross-sectional area of the flow path at the narrowed part is smaller than a cross-sectional area of the flow path at a part other than the narrowed part.

### Explanation of References

1: endoscopic treatment instrument

20: treatment member

21: treatment part

21C: opening and closing shaft

21A, 21B: claw part

21Aa, 21Ba: overlapping part

21a: serration

22: support part

23: connection part

23A: link rod

23B: cylindrical member

23C: operation wire

23D: coil sleeve

23E: covering member

221As, 221Bs, 221Cs: outer peripheral surface

30: housing member

30h: distal end side opening

30s: inner peripheral surface

40: distal end member

40A: connector member

40Ah: liquid supply part

40C, 40E: O-ring

40D: connector member

40F: fixing member

41A: connection terminal

41: cable connection part

42: slider

42A: wire fixing part

43: finger rest part

43A: cavity

50: operating part

100: liquid supply device

101: supply pipe

200: high-frequency power supply

201, 202: cable

203: counter electrode

221: shaft support member

221A: upper shaft support part

221B: lower shaft support part

221C: annular part

221D: extension part

221E, 221F: groove

221G: hole

222: coupling member

SP1, SP2, SP3, SP4, SP5, SP6: gap

What is claimed is:

1. An endoscopic treatment instrument comprising:
a treatment member including, on a distal end side, a treatment part that is openable and closable and a support part that supports a proximal end side of the treatment part, and extending in a first direction;
a housing member that has a cylindrical shape, that extends in the first direction, and into which the treatment member is inserted;
a flow path that is formed between an inner peripheral surface of the housing member and the treatment member and through which a liquid supplied from a proximal end side of the housing member to an inner portion of the housing member is capable of being discharged from a distal end side opening of the housing member; and
a narrowed part of the flow path,
wherein the narrowed part is provided on a distal end side with respect to a proximal end edge of the support part.

2. The endoscopic treatment instrument according to claim 1,
wherein the narrowed part is formed by the support part.

3. The endoscopic treatment instrument according to claim 2, further comprising:
an operating part that is provided on a proximal end side and that performs an opening and closing operation of the treatment part,
wherein the treatment member includes a connection part that connects the treatment part to the operating part, and
the support part includes a shaft support member that supports an opening and closing shaft of the treatment part and a coupling member that couples the shaft support member to the connection part.

4. The endoscopic treatment instrument according to claim 3,
wherein the narrowed part is formed by the shaft support member.

5. The endoscopic treatment instrument according to claim 4,
wherein the shaft support member has an outer peripheral surface facing the inner peripheral surface of the housing member,
the shaft support member is provided with a hole or a groove that extends in the first direction,
the shaft support member covers a part of the flow path on a proximal end side with respect to the shaft support member as viewed in the first direction from a distal end side, and
the narrowed part is formed by the outer peripheral surface and the hole or the groove of the shaft support member.

6. The endoscopic treatment instrument according to claim 5,
wherein the shaft support member includes shaft support parts that are arranged in a direction intersecting an opening and closing direction of the treatment part and that support the opening and closing shaft of the treatment part, and an annular part that is provided at proximal ends of the shaft support parts, and
the hole or the groove is provided in the annular part.

7. The endoscopic treatment instrument according to claim 5,
wherein the shaft support member includes shaft support parts that are arranged in a direction intersecting an opening and closing direction of the treatment part and that support the opening and closing shaft of the treatment part, and an annular part that is provided at proximal ends of the shaft support parts, and
the hole or the groove is provided in the shaft support part.

8. The endoscopic treatment instrument according to claim 3,
wherein the narrowed part is formed by the coupling member.

9. The endoscopic treatment instrument according to claim 1,
wherein the narrowed part is formed by the treatment part.

10. The endoscopic treatment instrument according to claim 9,
wherein the treatment part includes a claw part,
the claw part has an overlapping part that overlaps the flow path on a proximal end side with respect to the claw part as viewed in the first direction from a distal end side, and
the narrowed part is formed by the overlapping part.

11. The endoscopic treatment instrument according to claim 1,
wherein a cross-sectional area of the flow path at the narrowed part is smaller than a cross-sectional area of the flow path at a part other than the narrowed part.

12. The endoscopic treatment instrument according to claim 2,
wherein a cross-sectional area of the flow path at the narrowed part is smaller than a cross-sectional area of the flow path at a part other than the narrowed part.

13. The endoscopic treatment instrument according to claim 3,
wherein a cross-sectional area of the flow path at the narrowed part is smaller than a cross-sectional area of the flow path at a part other than the narrowed part.

14. The endoscopic treatment instrument according to claim 4,
wherein a cross-sectional area of the flow path at the narrowed part is smaller than a cross-sectional area of the flow path at a part other than the narrowed part.

15. The endoscopic treatment instrument according to claim 5,
wherein a cross-sectional area of the flow path at the narrowed part is smaller than a cross-sectional area of the flow path at a part other than the narrowed part.

16. The endoscopic treatment instrument according to claim 6,
wherein a cross-sectional area of the flow path at the narrowed part is smaller than a cross-sectional area of the flow path at a part other than the narrowed part.

17. The endoscopic treatment instrument according to claim 7,
wherein a cross-sectional area of the flow path at the narrowed part is smaller than a cross-sectional area of the flow path at a part other than the narrowed part.

18. The endoscopic treatment instrument according to claim 8,
wherein a cross-sectional area of the flow path at the narrowed part is smaller than a cross-sectional area of the flow path at a part other than the narrowed part.

19. The endoscopic treatment instrument according to claim 9,
wherein a cross-sectional area of the flow path at the narrowed part is smaller than a cross-sectional area of the flow path at a part other than the narrowed part.

20. The endoscopic treatment instrument according to claim 10,
wherein a cross-sectional area of the flow path at the narrowed part is smaller than a cross-sectional area of the flow path at a part other than the narrowed part.
